(19) United States
(12) Patent Application Publication
(10) Pub. No.: US 2025/0089951 A1
(43) Pub. Date: Mar. 20, 2025

(54) Portable Hand Dryer

(71) Applicants: Mari Regina Bory, La Verne, CA (US); Christopher Gregory Bory, La Verne, CA (US)

(72) Inventors: Mari Regina Bory, La Verne, CA (US); Christopher Gregory Bory, La Verne, CA (US)

(21) Appl. No.: 18/468,702

(22) Filed: Sep. 16, 2023

Related U.S. Application Data
(60) Provisional application No. 63/577,564, filed on May 4, 2023.

(51) Int. Cl. A47K 10/48; A61L 2/00; A61L 2/24; A61L 2/26

(52) U.S. Cl. CPC A47K 10/48; A61L 2/0047; A61L 2/24; A61L 2/26; A47K 2201/02; A61L 2202/11; A61L 2202/14; A61L 2202/16

(57) ABSTRACT

A portable hand dryer easily and removably attachable to a power outlet without tools, comprising a rectangular housing which retains electric power interface and control system devices. Prongs that plug into an electrical outlet receptacle bring electric power to the dryer protrude from the rear of rectangular housing. The hand dryer further comprises drying air and disinfecting UV light movably aimable housing. The housings are joined by a two degrees of rotational freedom swivel assembly joining the drying air and disinfecting housing. The hand dryer is securely held on a wall outlet by a combination of frictional forces of prongs inserted into a socket and mechanical or magnetic snap devices. The drying and disinfecting is automatic, initiated by a user proximity sensors disposed in the drying air and disinfecting UV light housing.

### CROSS REFERENCE TO RELATED APPLICATIONS

Not Applicable.

### STATEMENT REGARDING FEDERALLY SPONSORED RESEARCH OR DEVELOPMENT

Not Applicable.

### DESCRIPTION OF ATTACHED APPENDIX

Not Applicable.

### BACKGROUND OF THE INVENTION

This invention relates generally to the field of electrical hand dryers and more specifically to portable hand dryers that can be temporarily secured to an electrical wall outlet without tools and adjusted to a variety of angles for aiming drying air, typically hot, at a user's hands.

Electrically heated hand dryers are a well-known form of drying hands after washing them in a sink. These types of hand dryers are usually found in commercial bathrooms located in offices, restaurants and other community locations and use an electric motor and fan to blow air past a heating element and out onto the user's hands.

One version of an electric hand dryer was proposed in U.S. Pat. No. 6,766,589 invented by Maria Regina Bory et al. which was intended to be used in the bathroom of a home or of an office. The U.S. Pat. No. 6,766,589 version plugs into a standard wall socket.

Most hand dryers are too bulky to be easily portable, consequently are permanently mounted and hard wired. Relatively easily portable dryers are primarily intended to be used as hair dryer requiring a person to hold it in one hand while drying, therefore true sanitizing is not achievable by one person as the first holding hand is still wet thus making the dryer unsanitary.

Small plugin portable hand dryers also lack provisions for holding securely while plugged into a wall socket.

Moreover, small plugin portable hand dryers also lack sanitizing ultraviolet (UV) features.

### BRIEF SUMMARY OF THE INVENTION

This summary introduces in a simplified form the inventive concepts further disclosed in the DETAILED DESCRIPTION of the embodiments.

An object of the instant invention is to provide a portable hand dryer that can be easily removably secured to a bathroom wall via mating electric contacts of sockets and plugs, and also via additional fastening methods like snap type or magnetic fasteners thus overcoming the features lacking in the so far known hand dryers described in the BACKGROUND OF THE INVENTION above.

Disclosed in the detailed description of the preferred embodiment is a hand dryer assembly comprising three main portions. The first main portion is disposed in a rectangular housing which retains electric power interface and control system devices. Protruding from the rear of the first main housing are the prongs that plug into an electrical outlet receptacle, typically an electrically grounded receptacle in a wall mounted outlet. The dryer is held onto the wall mounted outlet by a combination of the dryer geometry when plugged in and the frictional forces of the electrical prongs when mated with corresponding sockets. As will be described in the detailed description, the rear portion of the instant invention comprises additional means, in addition to the electrical prongs-to-socket connection, to removably and securely attach the hand dryer to the wall.

The second main portion that retains a drying air moving fan and a drying air heating assembly, heated air is discharged out of the front of the second main portion housing. Housed in the second main portion is at least one motion detector and at least one source of disinfecting ultraviolet light aimed at the dryer user's hands during the hand drying process.

The third main portion is a swivel joint that allows the second main portion to be pointed at the user's hands regardless of the location or position of the electrical socket into which the hand dryer is plugged in and which removably supports the hand dryer.

The swivel joint includes a central hollow channel that allows electrical wires to pass to and from the fan motor, the heating assembly, the motion sensors, and ultraviolet light sources to the rectangular housing containing the electric power interface and control system devices.

### BRIEF DESCRIPTION OF THE DRAWINGS

The drawings constitute a part of this specification and include exemplary embodiments of the instant invention, which may be embodied in various forms. It is to be understood that in some instances various aspects of the instant embodiments may be shown exaggerated or enlarged to facilitate an understanding of the instant exemplary embodiments.

FIG. 1 is a front perspective view of the embodiment.

FIG. 2 is a rear perspective view of the embodiment using snap fasteners.

FIG. 3 is a side section view of the embodiment using snap fasteners.

FIG. 4 is an exploded view of the first main portion housing of the embodiment using snap fasteners about to be plugged in into and attached to a wall socket.

FIG. 5 is an exploded view of the embodiment.

FIG. 6 is a rear perspective view of the embodiment using magnetic fasteners.

FIG. 7 is a side section view of the embodiment using magnetic fasteners.

FIG. 8 is an exploded view of the first main portion housing of the embodiment using magnetic fasteners about to be plugged in and attached to a wall socket.

FIG. 9 is an exploded view of a version with a mechanism to assure the swivel assembly to hold in the user selected vertically rotational position.

FIG. 10 is a perspective view of the embodiment using a vertical hold adjuster.

### DETAILED DESCRIPTION OF THE PREFERRED EMBODIMENTS

Detailed descriptions of the preferred embodiments are provided herein. It is to be understood, however, that the present invention may also be embodied in other forms retaining the principles disclosed herein. Therefore, specific details disclosed herein are not to be interpreted as limiting, but rather as a basis for the claims and as a representative basis for teaching one skilled in the art to employ the present invention in virtually any appropriately detailed system, structure, or manner.

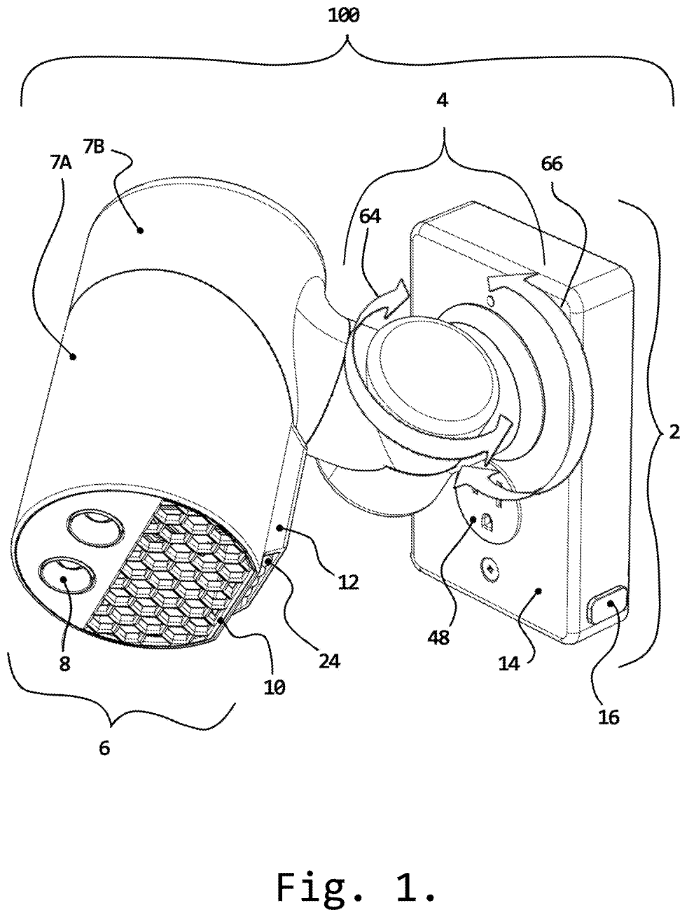

Referring now to FIG. 1 we see a perspective view of the portable hand dryer 100. It consists of three main portions: a rectangular housing 2 that plugs into a socket of an electrical wall outlet, a two degrees of rotational freedom adjustable swivel assembly 4, and a hand drying air heating, air moving, and disinfecting assembly 6, the assembly 6 comprised of two cylindrical housings 7A and 7B which house the air heating and moving components, and an under-housing 12 which comprises disinfecting and motion sensing components. The hand drying air heating, air moving, and disinfecting assembly 6 while being cylindrical in the instant embodiments can be implemented in a variety of shapes, regular as well as irregular ones. The shape of the hand drying air heating, air moving, and disinfecting assembly is also a function of esthetics and/or packaging for shipping and display.

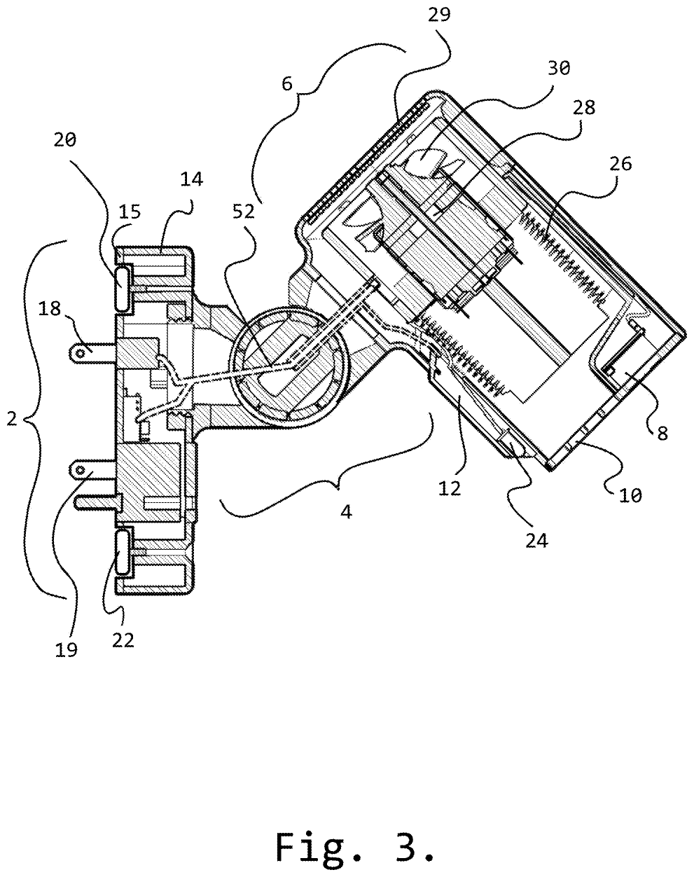

Housing 2 is essentially a rectangular cuboid comprised of six panels enclosing a hollow space. Even though the outside facing edges of the instant embodiment are shown beveled, the edges of the housing 2 can be of various shape for esthetical reasons, for example rounded. Further visible on a side panel is the electrical overload protection reset button 16 to be used to reset the electrical overload protection after an overload has been sensed by the control system. Motion sensor 8 detects the presence of a hand or hands and activates the fan motor 28, and the heating coil 26, and ultraviolet light emitting diodes (UV LEDs) 24 shown in FIG. 3, to start the drying process. The under-housing 12, attached to the underside of cylindrical portion 7 comprises an ultraviolet (UV) light generator, the UV generator in the instant embodiment comprises a plurality of UV LEDs 24 which shine outward towards the user's hands when the hand drying process is taking place. Adjustable swivel assembly 4 allows for up and down movement, depicted with arrows 64, as well as clockwise and counterclockwise rotational movement depicted with arrows 66 allowing the heating, disinfecting, and air moving assembly 6 to be positioned in an ideal location with respect to the user's hands no matter what the orientation of the wall outlet is. It is obvious to anyone knowledgeable in the art of hand dryers that adjustable stops can be added to limit rotation and/or swivel of the cylindrical shell.

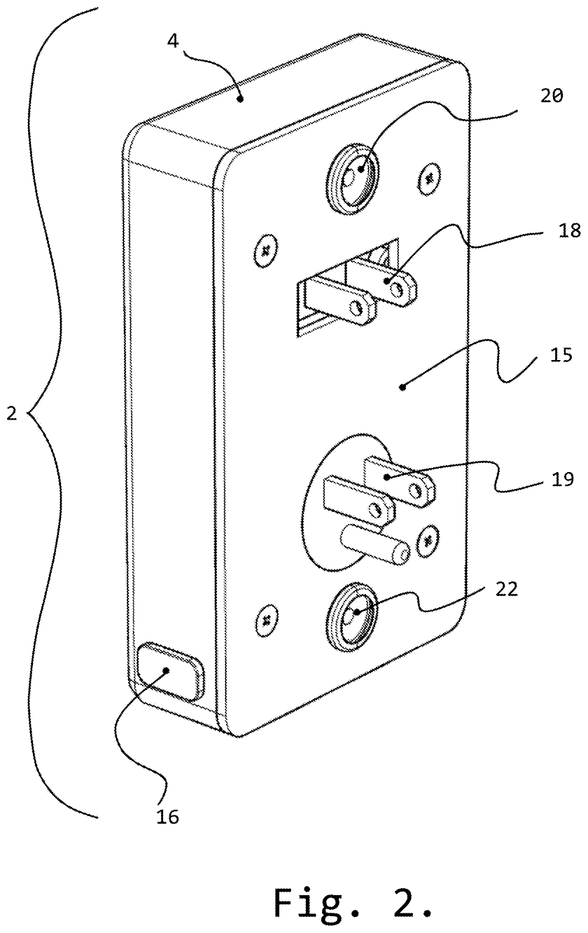

FIG. 2 is a perspective view of rear panel 15 of housing assembly 2. Electrical prongs 18, 19 extend outward from the rear panel 15 and are spaced to be able to plug into a most often used United States standard 15 A (amperes), 120 VAC wall socket 36 of an outlet assembly 40. The standard 120 VAC is sometime also referred to as either US standard 110 VAC, or 120 VAC (“VAC” is a frequently used abbreviation meaning “volts alternating current”). The 15 A, 120 VAC outlet version is depicted, however, FIG. 2 is not to be interpreted as limiting the instant embodiment of the invention to the 15 A, 120 VAC version. The instant invention design is easily adapted to most voltages and outlet configurations worldwide. The two-prong connection 18 shown in the instant embodiment can easily be adapted to a two-prong plus ground prong configuration.

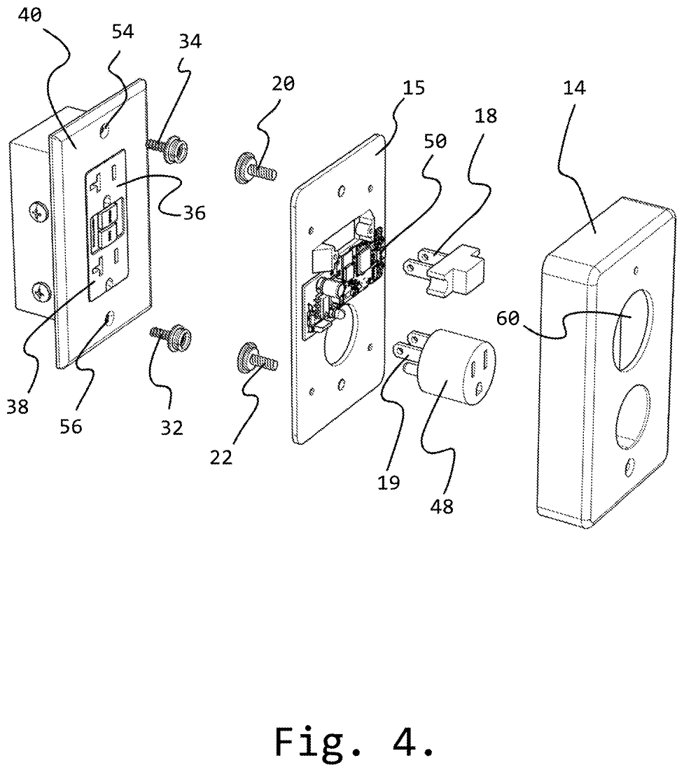

In order to securely retain the housing assembly 2 to the wall outlet assembly 40, female snap fasteners 20, 22 can removably engage mating male snap fasteners 32, 34 as shown in FIG. 3 and FIG. 4 and will be described in more detail when describing FIGS. 3 and 4. The male treads of snap fasteners 32, 34 match the female threads of a wall outlet meant to hold the outlet cover plate onto the main body outlet, allowing the male snap fasteners 32, 34 to be easily screwed in by the user. The snap fasteners 20, 22 hold the entire assembly 100 securely on the wall outlet relieving the strain that would otherwise be put on the electrical prongs 18, 19 that are normally meant to hold only a lightweight male plug and cord. The entire hand dryer 100 can be removed when needed by unsnapping the fasteners 20, 22.

FIG. 3 is a side section view of hand dryer 100. Prongs 18 are plugged into outlet sockets 40 to supply power to the portable hand dryer. A plug outlet extender 48 within the main housing 2, comprising prongs 19 that plug into a typical second 120 VAC socket 38; the extender can be used to plug in any other device that runs on 120 VAC. Furthermore, the extender can be removed for instances when used where only a single socket outlet is available.

Female snap fasteners 22, 20 are secured by threaded portions within the back panel 15 of the main housing 2 and can be seen engaging male snap heads of screws 32, 34. Another option for securing the hand dryer 100 to a wall outlet is to remove female snap screws 20, 22 and use long screws that enter the outside apertures 54, 56 shown in FIG. 4 and to screw those screws into the standard threaded apertures 36, 38 shown in FIG. 4. This method of attachment is most secure but requires additional screwdriver activity to install or remove the entire hand dryer 100.

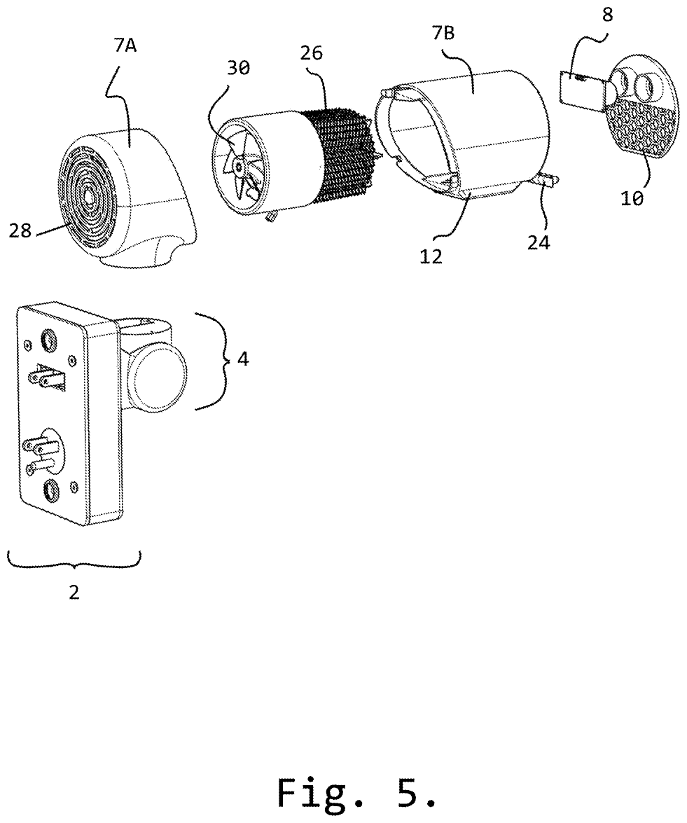

FIG. 5 depicts the motion detector, signals from a sensor 8, a sonar type sensor 8 is shown in the instant embodiment, travel through a hollow portion of adjustable swivel assembly 4 by wires 52 into the microcontroller 50. Further depicted in FIG. 5 are a perforated panel 28 allows air to flow into the 7A and 7B cylinder parts and be drawn in by fan blade 30 that is powered by motor 28. The fan blade 30 forces air over heating coil 26. Warm air then exits the front grilled panel 10 and out to be used to dry a person's hands. Sonar sensor 8 is designed to be sensitive to moving objects within two feet of the warm air exit grilled panel 10, causing motor 28, heater 26 and UV LEDs 24 to be powered while the user's hands remain in close proximity to the outlet of main cylinder 7A. The UV LEDs 24 emit a frequency of approximately two hundred and sixty nanometers which is known to kill germs and bacteria.

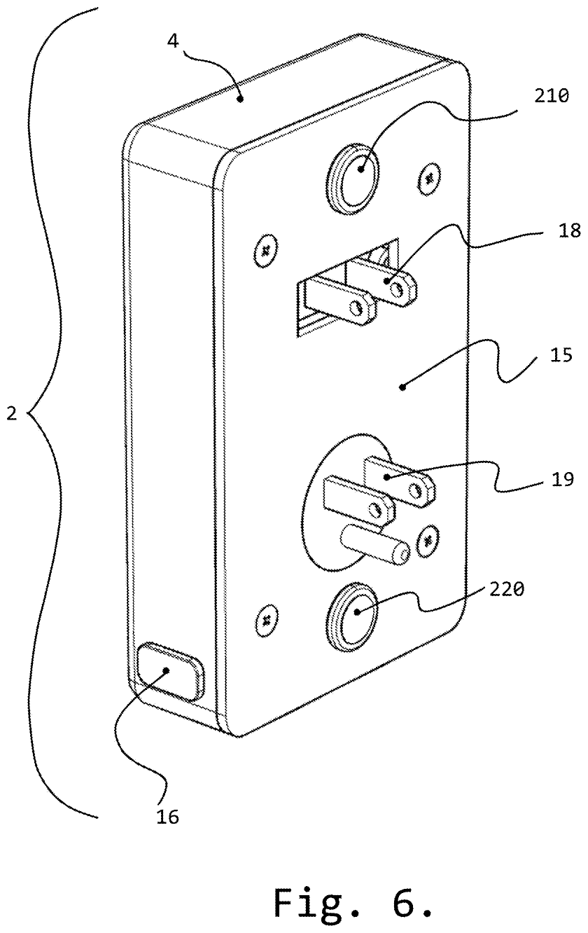

Shown in FIG. 6 Is an alternate embodiment wherein magnets mounted flush with the face of panel 15 facilitate the easy attachment and removal of the dryer 100 to the wall mounted outlet 40. Magnets 210 and 220 magnetically attach to fastening screws 540, 560 having a flat ferrous metal head, designed to screw into the standard threaded openings 54, 56 of a 120 VAC outlet as shown in FIG. 4, instead of the snap fasteners.

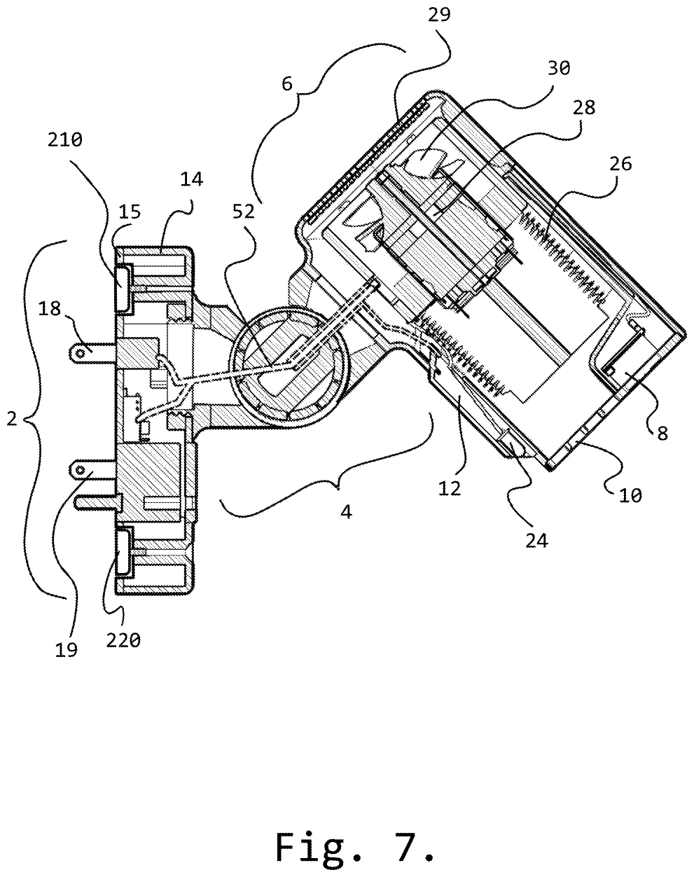

FIG. 7 is a side section view of hand dryer 100. Prongs 18 are plugged into outlet sockets 40 to supply power to the portable hand dryer. A plug outlet extender 48 within the main housing 2, comprising prongs 19 that plug into a typical second 120 VAC socket 38; the extender can be used to plug in any other device that runs on 120 VAC. Furthermore, the extender can be removed for instances when used where only a single socket outlet is available. Magnets 210, 220 are secured to the back panel 15 of the main housing 2 and can be seen engaging heads of screws 32, 34.

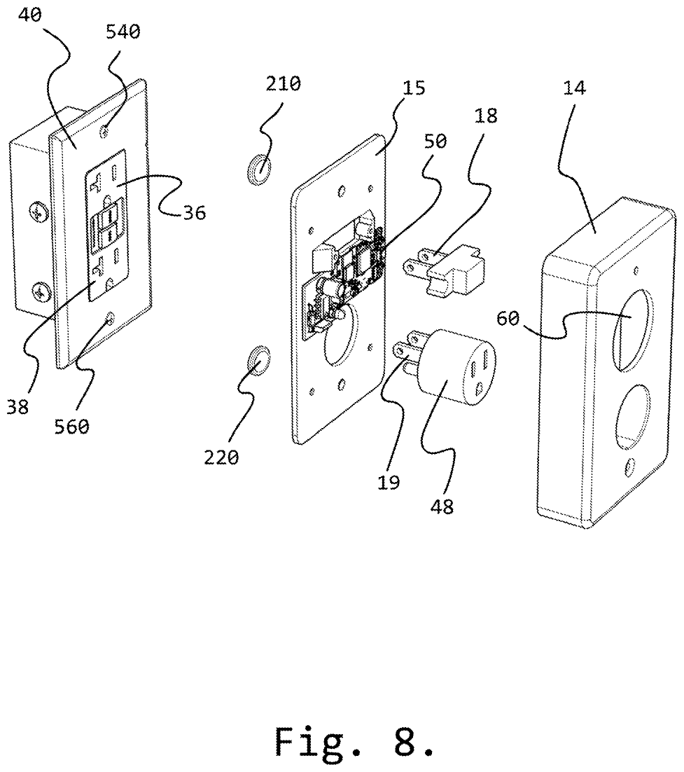

FIG. 8 is an exploded view of the magnetically attachable hand dryer 100.

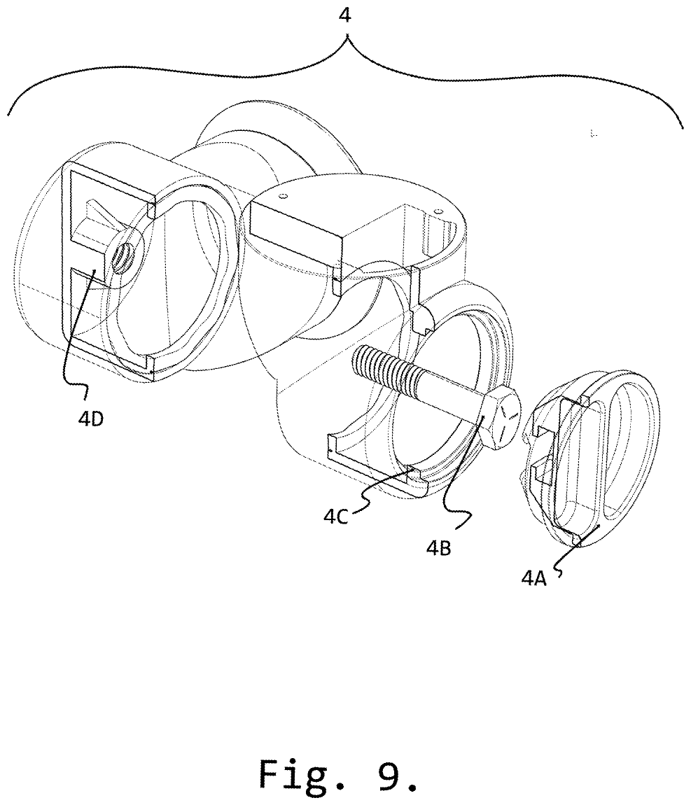

FIG. 9 is an exploded view of a version with a mechanism to assure the swivel assembly to hold in the user selected vertically rotational position. Bolt 4B threads into a threaded socket 4D while pressing the faces 4A of the circular bolt adjuster and of the swivel 4B together to increase or decrease the friction between the face 4B and the adjuster 4A, thus securing or releasing the swivel to hold the desired position.

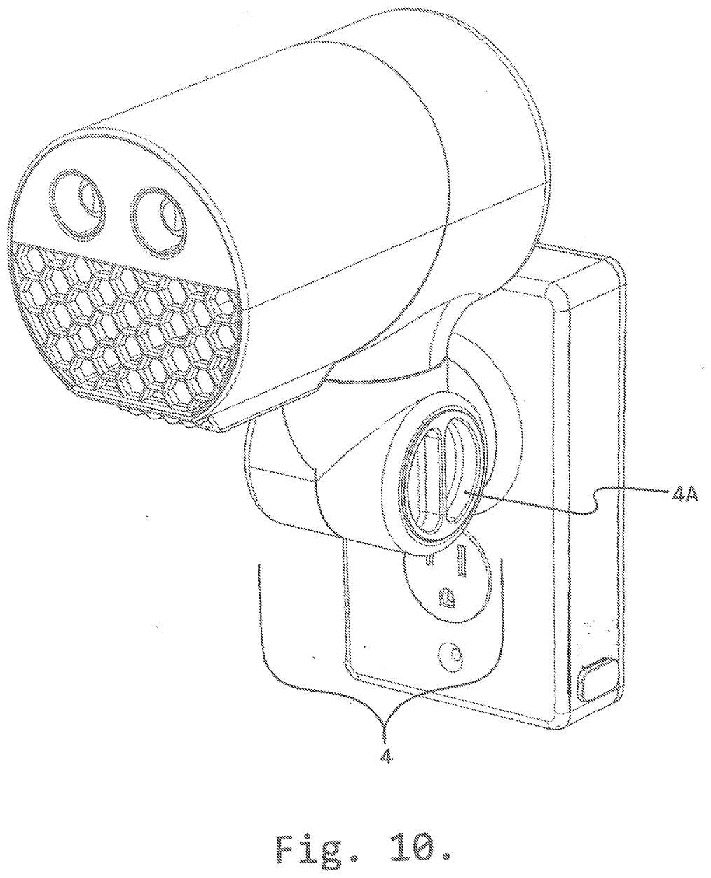

FIG. 10 shows the hair dryer with the adjuster 4A located on a side face of the .[00] In the claims, the word “comprising” does not exclude the presence of other elements or steps than those listed in a claim. Furthermore, the terms “a” or “an,” as used herein, are defined as “one, or more than one.” Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are arbitrarily used to distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

What is claimed is:

1. A portable removably snap attachable hand dryer comprising:
a first housing;
a second housing;
disposed in the first housing are: a plurality of attachment devices for removably attaching the portable hand dryer to a wall mounted electrical power outlet and a plurality of electrical power interface and control devices;
wherein the second housing being pivotally joined to the first housing by a compound swivel joint assembly, whereby the compound swivel assembly enables the second housing to rotate around a first axis perpendicular to the wall mounted electrical power outlet, and around a second axis, the second axis being perpendicular to the first axis and parallel to the wall mounted electrical power outlet;
disposed in the second housing are:
a fan and a heating assembly;
a user proximity sensor components assembly; and
a contactless hand disinfecting treatment components assembly.

2. The portable hand dryer of claim 1, wherein the first housing being a rectangular cuboid enclosing a hollow space wherein sides of the rectangular cuboid are comprised of a front panel, a rear panel, and a plurality of side panels, each panel comprising an inside surface facing the enclosed hollow space and outside surface facing a space without the enclosed hollow space wherein:
the plurality of electrical power interface devices disposed in the hollow space comprise a plurality of outward pointing prongs extending through the rear panel, wherein the outward pointing prongs serve to bring electrical power to the portable hand dryer and to provide frictional force between the portable hand dryer prongs and female sockets of the wall mounted electrical power outlet;
at least one pair of removable snap attachments, each removable snap attachment pair comprising a hand dryer component and a mating wall mounted component, wherein each hand dryer snap attachment coupling component is disposed on the rear panel outside surface and the mating wall mounted component of the removable attachment coupling is disposed on the wall mounted electrical power outlet;
the plurality of control devices disposed within the enclosed hollow space comprise a programmable digital processing unit configured to control the hand dryer operation, the programmable digital processing unit being in electrical communication with a plurality of sensors, and a plurality of switches; and
wherein first housing rectangular cuboid edges selected from a group comprising: sharp, beveled, or round.

3. The portable hand dryer of claim 1, wherein the second housing is comprising a cylindrical shell, the cylindrical shell comprising an air inlet end and an air discharge end, whereby disposed within the cylindrical shell between the air inlet end, and the air discharge end is a fan comprised of an electrical motor propelling a fan blade assembly, whereby further disposed next to the fan is a heating assembly, wherein air entered and moved by the fan through the inlet end is heated in the heating assembly and discharged through the air discharge end.

4. The portable hand dryer of claim 1, wherein the user proximity sensor assembly comprises a sonar subsystem tuned to detect a user's hand motion about two feet or closer from the air discharge end.

5. The portable hand dryer of claim 1, wherein the contactless hand disinfecting treatment components assembly comprises a plurality of ultra-violet light emitting diodes radiating at a wavelength of about 260 nanometers in a direction parallel with air discharge from the air discharge end.

6. The portable hand dryer of claim 1, further comprising a wall mounted power outlet extender.

7. The portable hand dryer of claim 1, further comprising an adjustable vertical rotation holding position adjuster comprising:
a threaded socket disposed near intersection of the first and the second axes, the threaded socket having a longitudinal axis wherein the socket longitudinal axis overlaps the second axis;
a bolt comprising a head on a bolt first end and a threaded second end portion, the bolt disposed along the second axis being capable to be threaded into the threaded socket;
a circular bolt adjuster rotatable around the second axis, the circular bolt adjuster also serving as an aesthetic cover of a side of the compound swivel assembly, the bolt adjuster engaging the bolt head; and
wherein rotating the circular bolt adjuster moves the circular bolt adjuster closer to or further from the swivel joint assembly.

8. A portable removably magnetically attachable hand dryer comprising:
a first housing;
a second housing;
disposed in the first housing are: a plurality of attachment devices for removably attaching the portable hand dryer to a wall mounted electrical power outlet and a plurality of electrical power interface and control devices;
wherein the second housing being pivotally joined to the first housing by a compound swivel joint assembly, whereby the compound swivel assembly enables the second housing to rotate around a first axis perpendicular to the wall mounted electrical power outlet, and around a second axis, the second axis being perpendicular to the first axis and parallel to the wall mounted electrical power outlet;
disposed in the second housing are:
a fan and a heating assembly;
a user proximity sensor components assembly; and
contactless hand disinfecting treatment components assembly.

9. The portable hand dryer of claim 8, wherein the first housing being a rectangular cuboid enclosing a hollow space wherein sides of the rectangular cuboid are comprised of a front panel, a rear panel, and a plurality of side panels, each panel comprising an inside surface facing the enclosed hollow space and outside surface facing a space without the enclosed hollow space wherein:
the plurality of electrical power interface devices disposed in the hollow space comprise a plurality of outward pointing prongs extending through the rear panel, wherein the outward pointing prongs serve to bring electrical power to the portable hand dryer and to provide frictional force between the portable hand dryer prongs and female sockets of the wall mounted electrical power outlet;
at least one pair of removable attachment magnets, each removable magnet pair comprising a hand dryer component and a mating wall mounted component, wherein each hand dryer attachment coupling magnet is disposed on and flush with the rear panel outside surface and the mating wall mounted component of the removable attachment coupling being disposed on the wall mounted electrical power outlet;
the plurality of control devices disposed within the enclosed hollow space comprise a programmable digital processing unit configured to control the hand dryer operation, the programmable digital processing unit being in electrical communication with a plurality of sensors, and a plurality of switches; and
wherein first housing rectangular cuboid edges selected from a group comprising: sharp, beveled, or round.

10. The portable hand dryer of claim 8, wherein the second housing is comprising a cylindrical shell, the cylindrical shell comprising an air inlet end and an air discharge end, whereby disposed within the cylindrical shell between the air inlet end, and the air discharge end is a fan comprised of an electrical motor propelling a fan blade assembly, whereby further disposed next to the fan is a heating assembly, wherein air entered and moved by the fan through the inlet end is heated in the heating assembly and discharged through the air discharge end.

11. The portable hand dryer of claim 8, wherein the user proximity sensor assembly comprises a sonar subsystem tuned to detect the user's hand motion about two feet or closer from the air discharge end.

12. The portable hand dryer of claim 8, wherein the contactless hand disinfecting treatment components assembly comprises a plurality of ultra-violet light emitting diodes radiating at a wavelength of about 260 nanometers in a direction parallel with air discharge from the discharge end.

13. The portable hand dryer of claim 8, further comprising a power outlet extender of a wall mounted power outlet not occupied by the hand dryer.

14. The portable hand dryer of claim 8, further comprising an adjustable vertical rotation holding position adjuster comprising:
a threaded socket disposed near the intersection of the first and second axes, the threaded socket having a longitudinal axis wherein the socket longitudinal axis overlaps the second axis;
a bolt comprising a head on a bolt first end and a threaded second end portion, the bolt disposed along the second axis being capable to be threaded into the threaded socket;
a circular bolt adjuster rotatable around the second axis, the circular bolt adjuster also serving as an aesthetic cover of a side of the compound swivel assembly, the bolt adjuster engaging the bolt head;
wherein rotating the circular bolt adjuster moves the circular bolt adjuster closer to or further from the swivel joint assembly.
